(19) United States
(12) Patent Application Publication
(10) Pub. No.: US 2002/0162128 A1
(43) Pub. Date: Oct. 31, 2002

(54) Non-human animal deficient in function of pituitary adenylate cyclase-activating polypeptide gene

(76) Inventors: Akemichi Baba, Nishinomiya-shi (JP); Toshio Matsuda, Osaka (JP); Hitoshi Hashimoto, Osaka (JP); Norihito Shintani, Osaka (JP)

Correspondence Address: ARMSTRONG,WESTERMAN & HATTORI, LLP, 1725 K STREET, NW., SUITE 1000, WASHINGTON, DC, 20006, US

(21) Appl. No.: 10/073,135

(22) Filed: Feb. 13, 2002

Related U.S. Application Data
(63) Continuation-in-part of application No. 09/835,627, filed on Apr. 17, 2001, now abandoned.

(30) Foreign Application Priority Data
Apr. 19, 2000 (JP) 2000-118088

(51) Int. Cl. A01K 67/027

(52) U.S. Cl. 800/9; 800/18

(57) ABSTRACT

A mammalian model animal for psychiatric disorders having a chromosome of a somatic cell and a germ cell with deficiency of function of pituitary adenylate cyclase-activating polypeptide gene.

### CROSS-REFERENCE TO RELATED APPLICATION

The present application is a continuation-in-part of U.S. patent application Ser. No. 09/835,627 filed Apr. 17, 2001, the content of which is incorporated herein in its entirety by reference.

### BACKGROUND OF THE INVENTION

1. Field of the Invention

The present invention relates to a non-human model animal for psychiatric disorders with deficient in function of pituitary adenylate cyclase-activating polypeptide (hereinafter referred to as PACAP) gene. More particularly, the present invention relates to a non-human animal having a chromosome of a somatic cell and a germ cell with deficiency of function of PACAP gene.

2. Description of the Related Art

PACAP is a neuropeptide isolated from ovine hypothalamus on the basis of its ability to stimulate adenylate cyclase in cultured rat anterior pituitary cells. PACAP plays an important role as peptide hormone, neurotransmitter or neuromodulator in the central nervous system and the peripheral nervous system. For example, the facts that the expression of PACAP is increased in cerebral ischemic accident or that intracerebral administration of PACAP can suppress nerve cell death caused by such accident are demonstrated. Further, since expression of PACAP in the nerve cells is strongly increased as a result of impairment of peripheral nerve such as neurotmesis, PACAP is suggested to be an essential factor for maintaining survival of nerve cells and recovery of the impaired nerve function.

It is further reported that PACAP strongly stimulates glucose dependent insulin secretion from the pancreas. In addition, it is reported that PACAP can be involved in development of gastric ulcer. As described above, the possibility of involvement of PACAP in important physiological functions in tissues expressing PACAP has been reported. The mouse PACAP gene, a gene coding PACAP, has already been isolated by the present inventors, and the total primary sequence thereof has been elucidated (Yamamoto, et al., Gene, 211, 63-69 (1998)).

To elucidate what actions PACAP, a specific polypeptide, itself elicits on animals is, however, quite difficult, since the impairment and degeneration in the above central nervous system and the peripheral nervous system, and the pathophysiology of the above various diseases are actually mixture of complex factors.

### SUMMARY OF THE INVENTION

The present inventors have studied extensively to solve these problems. As a result, they have found that if a line of mutant animal, which was deficient in PACAP gene, genetically stable and obvious in genetic background, could be obtained, it would be quite useful as an experimental animal for studies on the physiological functions of PACAP itself as well as the pathophysiology of the above diversified diseases to which PACAP pertains, elucidation of the etiology and development of drugs for treatments, and completed the present invention. At present, no animal that is genetically stable with deficiency of function of PACAP gene is known.

The present inventors have generated mice deficient in PACAP (hereinafter referred to as PACAP−/− mice or PACAP knockout mice) in order to understand the in vivo function of PACAP-dependent signaling and discovered a new role for PACAP in the regulation of psychomotor functions, and found that dysfunction of PACAP-mediated signaling pathways can lead to the pathologic changes characteristic of certain psychiatric disorders.

More specifically, the present inventors have found that PACAP−/− mice display markedly increased locomotor activity, novelty-related exploration, and explosive jumping behavior. This aberrant behavior is ameliorated by the antipsychotic drug haloperidol. Finally, the serotonin (5-HT) metabolite 5-hydroxyindoleacetic acid (5-HIAA) is slightly decreased in the cortex and striatum of the PACAP−/− mouse brain. The present inventors provides evidence that PACAP plays a new role in the regulation of psychomotor behaviors.

An object of the present invention is to provide a non-human model animal for psychiatric disorders which is deficient in the function of PACAP gene.

Particularly, an object of the present invention is to provide the above non-human model animal, especially rodents such as mouse for psychiatric disorders, which has a chromosome of a somatic cell and a germ cell with deficiency of function of PACAP gene.

Namely, the present invention is as follows:

(1) A mammalian model animal for psychiatric disorders having a chromosome of a somatic cell and a germ cell with deficiency of function of pituitary adenylate cyclase-activating polypeptide gene.

(2) The mammalian model animal according to the above (1), wherein the function is defective due to deficiency of a part or whole of exon 5 in the pituitary adenylate cyclase-activating polypeptide gene.

(3) The mammalian model animal according to the above (1), wherein the function is defective due to introducing a point mutation or inserting another gene in exon 5.

(4) The mammalian model animal according to the above (2), wherein a part or whole of exon 5 is deleted by substituting the part or whole of the exon 5 by another gene.

(5) The mammalian model animal according to the above (4), wherein the another gene is a marker gene.

(6) The mammalian model animal according to the above (5), wherein the marker gene is a neomycin resistance gene.

(7) The mammalian model animal according to the above (1), wherein the mammalian animal is a rodent.

(8) The mammalian model animal according to the above (1), wherein the mammalian animal is a mouse.

The animal of the present invention which is deficient in the function of PACAP gene due to a genetically engineering technique is quite useful as an experimental animal for studies on the psychiatric disorder, the elucidation of biological mechanism and development of drugs for treatment of the diseases.

### BRIEF DESCRIPTION OF THE DRAWING

FIG. 1A shows alignment of the PACAP locus with the targeting vector and the mutant locus.

FIG. 1B shows Southern blot analysis of tail DNA digested and hybridized.

FIG. 1C shows in situ hybridization analysis of parasagittal brain sections.

FIG. 1D shows RT-PCR analysis of RNA from midbrain and diencephalon.

FIG. 1E shows analysis of PACAP38 levels by radio immunoassay.

FIG. 2A shows total activity in open field measures.

FIG. 2B shows time spent in motion in open field measures.

FIG. 2C shows vertical activity in open field measures.

FIG. 3A shows the number of jumps in open field measures.

FIG. 3B shows time spent in the center in open field measures.

FIG. 3C shows center access, i.e. percentage of crossings into the center zone out of the total number of crossings in open field measures.

FIG. 3D shows examples of locomotor patterns of mice in open field measures.

FIG. 4A shows total activity to see effects of haloperidol on locomotor activity.

FIG. 4B shows catalepsy in the bar test to see effects of haloperidol on locomotor activity.

FIG. 5A shows tissue content of monoamines and their metabolites in cerebral cortex.

FIG. 5B shows tissue content of monoamines and their metabolites in striatum.

### PREFERRED EMBODIMENT OF THE INVENTION

In the present invention, the deficiency of the function of PACAP gene means that the structure of the gene is different from the naturally occurring structure and the gene cannot be expressed and PACAP cannot be produced. In order to obtain an animal which is artificially made deficient in the function of PACAP gene, the gene is cloned and the function of the gene is disrupted in vitro by any means, then the defective gene is transferred into the animal to make the animal itself or its progeny deficient in the function of the gene.

In the present invention, non-human animal(s) means any animals except for human, preferably mammalians such as rodents, more preferably mice.

Means for inserting an external gene into an animal and expressing the gene in the individual of the animal or its progeny include implanting into the animal a host embryo obtained by any of (1) inserting a genomic DNA into a pronuclear embryonic phase of a fertilized ovum, (2) infecting the early phase embryo of the animal with the recombinant retrovirus using a genomic DNA, and (3) using a genomic DNA as a targeting vector to construct a homologous recombination and inserting the embryonic stem cells (ES cells) of the animal into the blastocyst or 8-cell embryo, thereby obtaining offspring, which is mated up with another individual to prepare F1 heterozygote variant animals and further to prepare F2 homozygote variant animals. These means have been known in the art for preparing transgenic animals.

Among the above methods, the gene transfer using ES cells is preferable, since it has advantages that the process can be divided into a process for transferring a gene into ES cells and a process for preparing chimera animals. ES cells are considered to be able to be cultured in principle in general mammalians. In rats, rabbits and livestock (except mice) such as porcine and bovine, studies are in progress for establishment of ES cells. In the animal species in which method using ES cells are not established, the above method (1) or (2) can be used. Since a method for gene transfer using ES cells has been established especially in mice, the present invention will be explained using mice as an example, but the present invention is not limited to this example.

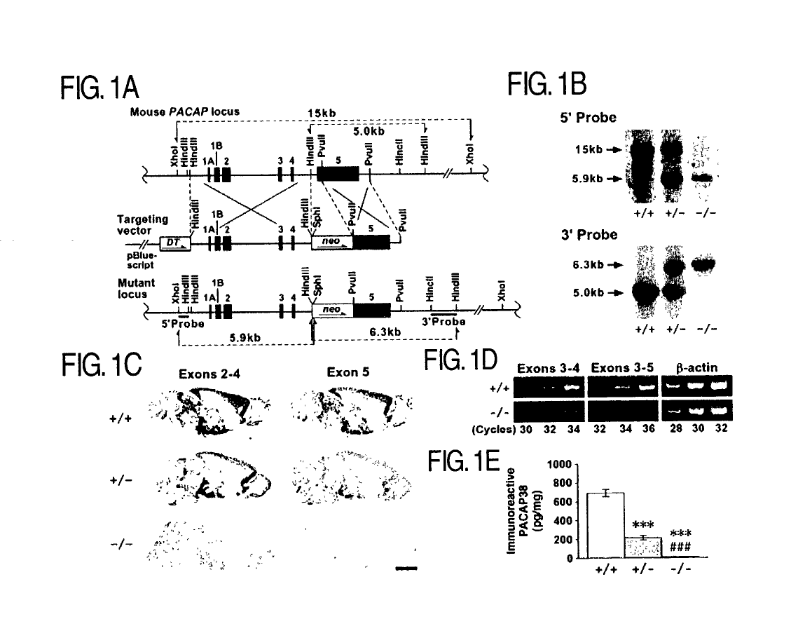

According to the description of the above reference, Yamamoto et al., Gene, 211, 63-69 (1998), the mouse PACAP gene spans 6.6 kb of the genomic DNA and is composed of six exons (exon 1A or exon 1B and exons 2-5), including two 5′-untranslated exons. Exons 1A and 1B encode the 5′-untranslated region (5′-UTR) of the PACAP DNA. Exon 2 encodes 5′-UTR, signal peptide and a part of amino-terminal peptide. Exon 3 encodes most of the amino-terminal peptide. Exon 4 encodes a PACAP related peptide (PRP). Exon 5 encodes the connecting peptide, mature PACAPs (PACAP-38 and PACAP-27), the carboxy-terminal peptide and the 3′-UTR, and contains two potential polyadenylation signals. The mouse PACAP gene encodes a PACAP precursor consisting of 175 amino acid residues in total from exon 2 to exon 5. Limiting decomposition of the precursor results in production of physiologically active PACAP-38 consisting of 38 amino acid residues or PACAP-27 consisting of 27 amino acid residues at the N-terminal side of PACAP-38. As described above, these PACAPs are encoded in a part of exon 5 in mouse PACAP gene (FIG. 1)

Consequently, if exon 5, preferably the function in the region encoding matured PACAPs on exon 5, is made defective, the mouse of the present invention which is deficient in the function of PACAP gene can be prepared. Examples of methods for preparing defective function of the region encoding matured PACAPs on exon 5 include the following: to delete a part or whole of the region, to insert a point mutation into the region, to insert another gene into the region and to substitute another gene for a part or the whole of the region to delete the above part or the whole of the region. It is preferable to select a proper marker gene as the another gene and substitute the proper marker gene for the region to construct a targeting vector (DNA for homologous recombination) for deficiency of the function of said region. At this time, the marker gene used is preferably such that a positive/negative selection can be performed in order to select the objective homologous recombinant.

A process for preparing a PACAP knockout mouse is described below.

Preparation of Targeting Vector of Mouse PACAP Gene (DNA for Homologous Recombinant)

In order to select the objective homologous recombinant and simultaneously make the function of PACAP gene defective, a positive/negative selection is performed. For these functional deficiency and selection, insertion of neomycin resistance gene (neo) and diphtheria toxin A fragment (DT) are especially preferable according to Yagi et al. (Yagi, Nada, Watanabe et al., Analytical Biochemistry, 214, 77-86 (1993)).

PACAP Gene Deficiency of ES Cells by Means of Homologous Recombination

The thus obtained DNA for the homologous recombination is suspended in a buffer such as phosphate buffer saline (PBS) containing mouse ES cells (e.g. E14 strain), and the gene is transferred into ES cells. Selective culture using G418 as a selective agent is performed for positive selection. Colonies resistant to the selective agent are examined by Southern blotting to confirm homologous recombinant. The thus obtained ES cells are injected into host embryos such as blastocyst or 8-cell embryo of a mouse of a proper line, and a chimera mouse is prepared by conventional method to obtain a PACAP knockout mouse in which the homologous recombinant ES cells are transferred into the germ cell line.

Behavioral Abnormality of PACAP−/− Mice

PACAP−/− mice exhibit profound behavioral abnormalities, including hyperactive locomotion, explosive jumping and reduced anxiety when placed in a novel environment. This hyperactivity is ameliorated by haloperidol. In the brain, 5-HT turnover is slightly decreased. These results provide evidence that PACAP plays a new role in the regulation of psychomotor behaviors.

Thus, PACAP−/− mice serve as a mammalian model animal for some aspects of the psychiatric disorders which include schizophrenia, emotional disturbance, bipolar affective disorder, hyperactivity disorder and the like.

### EXAMPLE 1

### Preparation of PACAP Knockout Mouse

(1) Preparation of DNA for Homologous Recombinant DNA in Mouse PACAP Genomic DNA

In order to disrupt the PACAP gene, 0.5 kb region encoding matured PACAPs on exon 5 was made defective by conventional method, and neomycin resistance gene was inserted therein and diphtheria toxin A fragment gene was inserted into the upstream of exon 1A. The homologous region with the genomic DNA was constructed to be 5.3 kb length between the diphtheria toxin A fragment gene and the neomycin resistance gene and 2.1 kb length of the downstream of the neomycin resistance gene. The thus obtained construct was inserted into pBluescript KS and split with restriction enzyme NotI for linearization at the time of transfer into ES cells, whereby a DNA for homologous recombination, a targeting vector, was obtained (FIG. 1).

(2) PACAP Gene Deficiency of ES Cells by Transferring DNA for Homologous Recombination

One hundred micrograms of DNA for homologous recombination was suspended in PBS (137 mM NaCl, 2.7 mM KCl, 10 mM Na₂HPO₄ and 1.8 mM KH₂PO₄) containing 3×10⁷ mouse ES cells (E14 strain), and a gene transfer was performed under the conditions of an electric field strength of 2 kV/cm and an electrostatic capacity of 3 μF. After 24 hours of the transfer, a selection culture was performed with 150 μg/ml of G418 (Geneticin, GIBCO BRL).

After 8 days of the gene transfer, G418 resistant colonies were transferred into 96 well microplate (IWAKI 3870-096) added with PBS containing 50 μl of 0.25% trypsin (GIBCO BRL) using a micro pipette, and treated for several minutes to prepare single cells by pipetting. These were transferred to 48 well plate (IWAKI 3830-048) and cultured. After 3-4 days, at the stage when cells on the 48 well plate reached confluent or were close to confluence, the cells were treated with 0.25% trypsin, then transferred to two wells on the 24 well plate (IWAKI 3820-024) and further cultured. After the cells were proliferated again, genomic DNA for Southern analysis was extracted from the cells in the one well of 24 well plate, and cells in another well were transferred into the 6 well plate (IWAKI 3810-006), cultured and then preserved in freezing.

The genomic DNA was extracted from G418 resistant cells, digested with restriction enzyme HindIII and then subjected to a Southern blotting analysis using as a probe 1.1 kb of a HincII-HindIII fragment in the downstream of exon 5. The homologous recombinant containing disrupted allele (FIG. 1) and non-homologous recombinant were confirmed by detecting bands of 6.3 kb and 5.0 kb, respectively. The number of recombinant colonies was 4 (clones 41, 44, 65-9 and 86) of 264 colonies of G418 resistant colonies.

(3) ES Cells and Culture Thereof

As for ES cells, E14 strain derived from 129/O1a mouse blastocyst was used. For culture of ES cells, ES culture medium consisting of Glasgow minimum essential medium (GMEM, GIBCO BRL) added with 10% fetal calf serum (FCS), 0.1 mM 2-mercaptoethanol, a non-essential amino acid solution, 1 mM sodium pyruvate and LIF (GIBCO BRL).

(4) Preparation of Chimera Mouse Using PACAP Deficient ES Cells

This was performed by conventional manner. After inserting the homologous recombinant clones 41 and 65-9 of ES cells into 15 blastocysts and 9 blastocysts of C57BL/6 mice, these blastocysts were implanted into uteri of recipient ICR strain, female, to obtain 4 and 2 offsprings, respectively. Among the respective delactated 2 offsprings, each one could be determined as chimera mouse by observing hair color, each of which was morphologically male. Rate of contribution of ES cells in these chimera mice was about 70-80%.

(5) Mating of Chimera Mice

Chimera mice obtained by the implantation were mated with C57BL/6 mice, female, and examined as to whether the obtained offsprings (F1 heterozygote mice) were originated from PACAP gene-defective ES cells or not was detected. If the germ cells of the chimera mouse are derived from the ES cells, the color of hair of the offsprings shows wild type color, and if it is derived from blastocysts of C57BL/6 mouse, the color of offsprings show black. Transfer of ES cells to germ cell line was confirmed in both of chimera mice originated from ES cell clones 41 and 65-9, respectively.

As the results of mating with chimera mouse derived from ES cell clone 41 and C57BL/6 mouse, female, total 51 offsprings were obtained from 6 times of deliveries. Among them, 45 mice survived and all showed wild color. As the results of mating with chimera mouse derived from ES cell clone 65-9 and C57BL/6 mouse, female, total 70 offsprings were obtained in 8 times of deliveries. Among them, 62 mice survived, further among the survivals, 21 mice showed wild color.

As a result of Southern blotting analysis of the wild color mice, 20 out of 45 mice derived from the clone 41, and 8 out of 21 mice derived from the clone 65-9, were confirmed to be deficient in the PACAP gene. Matings were further performed within F1 heterozygotes mice which were confirmed to be deficient in the gene, or within F2 heterozygotes mice obtained from mating between F1 heterozygote mouse and C57BL/6 mouse. As a result, 134 offsprings and 33 offsprings, obtained from clone 41 and clone 65-9, respectively, were obtained. Southern blotting analyses showed that F2 or F3 homozygote mice were deficient in the PACAP gene, in 25 mice and 4 mice from clone 41 and clone 65-9, respectively.

### EXAMPLE 2

### Altered Psychomotors Behaviors in PACAP Knockout Mice

(1) Materials and Methods

All animal care and handling procedures were approved by the institutional animal care and use committee of Osaka University.

(1-1) Generation of PACAP−/− Mice

The PACAP gene targeting vector was constructed from genomic DNA clones (λMPL4 and λMPL18) (Yamamoto, K., Hashimoto, H., Hagihara, N., Nishino, A., Fujita, T., Matsuda, T. & Baba, A. (1998) Gene 211, 63-69) isolated from a 129/SvJ mouse genomic library. A 2.1-kb PvuII fragment of the PACAP gene containing part of exon 5 and the 3′ flanking region was inserted 3′ to the neomycin resistant (neo) gene (derived from pGEM7-PGK-neo-polyA) in pBluescript KS(+). A MC1 promoter-driven diphtheria toxin A-fragment (DT) gene (derived from pMC1DTpA) was then inserted 5′ to the neo gene. Subsequently, a 5.3-kb HindIII genomic DNA fragment containing exons 1A through 4 was inserted between the DT and neo genes, to generate the PACAP targeting vector (FIG. 1A). The linearized vector was electroporated into 129/O1a mouse-derived E14tg2a ES cells. Targeted clones were identified by Southern blot analysis using external 0.42-kb and 1.1-kb probes and microinjected into C57BL/6 E3.5 blastocysts. Two highly chimeric males showed germ-line transmission and were mated with C57BL/6 wild-type females to produce Fl heterozygous mice. F1 heterozygotes were mated with C57BL/6 mice to produce the F2- and F3-generation mice, which were used in this study unless otherwise specified. The null allele of PACAP was also backcrossed 5 times with ICR mice, whose litter sizes were much larger than C57BL/6 mice. Wild-type mice and mice homozygous for the mutant PACAP gene were obtained from the intercross of heterozygous animals, and experiments were conducted with adult (three to five months old) mice. Reverse transcription-polymerase chain reaction (RT-PCR) was performed as described in Hashimoto, H., Hagihara, N., Koga, K., Yamamoto, K., Shintani, N., Tomimoto, S., Mori, W., Koyama, Y., Matsuda, T. & Baba, A. (2000) J. Neurochem. 74, 501-507, using the following PACAP gene exon-specific primers: exon 3, 5′-AGA AGA CGA GGC TTA CGA CCA G-3′ (sense); exon 4, 5′-ACG ACC GAC TGC AGG TAC TTC-3′ (antisense); and exon 5, 5′-TTT CTT GAC AGC CAT TTG TTT TCG G-3′ (antisense). The β-actin housekeeping gene was simultaneously reverse transcribed and amplified as previously described in Kitanaka, J., Hashimoto, H., Sugimoto, Y., Sawada, M., Negishi, M., Suzumura, A., Marunouchi, T., Ichikawa, A. & Baba, A. (1995) Biochim. Biophys. Acta 1265, 220-223. In situ hybridization analysis was performed on parasagittal brain sections as described in Hashimoto, H., Nogi, H., Mori, K., Ohishi, H., Shigemoto, R., Yamamoto, K., Matsuda, T., Mizuno, N., Nagata, S. & Baba, A. (1996) J. Comp. Neurol. 371, 567-577. Two different cDNA fragments of mouse PACAP (Yamamoto, K., Hashimoto, H., Hagihara, N., Nishino, A., Fujita, T., Matsuda, T. & Baba, A. (1998) Gene 211, 63-69), a 431-bp cDNA fragment (-116 to 315, where +1 represents the nucleotide position of the ATG initiation codon) spanning exons 2-4, and a 198-bp fragment (340 to 537) containing part of the exon 5 coding sequence deleted by homologous recombination, were used as templates to synthesize 35S-CTP-labeled cRNA probes. The expression of the biologically active mature PACAP isoform, PACAP38, was studied in brain by a radioimmunoassay kit (Peninsula Labs, Belmont, CA, USA).

(1-2) Open Field Test

Motor activity was quantified using an automated video-tracking system, the video image motion analyzer AXIS-90 (Neuroscience, Inc., Tokyo, Japan). A computer program was used to overlay grid lines defining 25 separate regions within a circular open field (60 cm in diameter, 30 cm deep, illuminated with white light, 100 lux). Paths taken by each mouse were stored permanently as x-y coordinate sequences, and parameters indicative of locomotor activity were assessed. Vertical activity (rearing and jumping) were also scored. In the haloperidol study, the drug was injected intraperitoneally 20 min prior to monitoring locomotor activity, which was measured for 90 min by another activity-monitoring system, Supermex (multi-channel system; Muromachi Kikai Co., Ltd., Tokyo, Japan). Subsequently, the same mice were tested for catalepsy in the bar test as previously described in Boulay, D., Depoortere, R., Oblin, A., Sanger, D. J., Schoemaker, H. & Perrault, G. (2000) Eur. J. Pharmacol. 391, 63-73.

(1-3) Elevated Plus Maze

In an elevated crossbar with two walled and two open arms, the movement of mice was recorded for 5 min. Also, the total path length, as well as the number of entries into and time spent on the open arms was assessed as described in Lister, R. G. (1987) Psychopharmacology Berl. 92, 180-185, using AXIS-90.

(1-4) Emergence Test

The emergence test was performed as previously described in Dulawa, S. C., Grandy, D. K., Low, M. J., Paulus, M. P. & Geyer, M. A. (1999) J. Neurosci. 19, 9550-9556, with several modifications. Briefly, the open field contained a white plastic cylinder with an open end (11 cm in diameter, 6 cm deep) located centrally. Mice were placed into the cylinder and tested for 15 min. A trained observer blind to genotype scored the following behaviors: the latency of emergence from the cylinder (defined as placement of all four paws into the open field); the total time spent inside the cylinder; and the exploratory behavior (assessed by frequency of rearing to the wall of the cylinder during the first 5 min).

(1-5) Novel Object Test

The novel object test was performed as previously described in Dulawa, S. C., Grandy, D. K., Low, M. J., Paulus, M. P. & Geyer, M. A. (1999) J. Neurosci. 19, 9550-9556, with several modifications. Briefly, mice were familiarized with the open field by preexposure to the open field for 2 days (day 1 for 60 min and day 2 for 30 min). On day 3, mice were placed in the open field for 30 min. Then, a novel white cup was placed into the center of the open field, and mice were tested for an additional 10 min. The number of entries made into the center with a diameter of 12 cm (center access) and time spent in the center were assessed by AXIS-90.

Measurements of Monoamine Neurotransmitters and Metabolites

Fresh-frozen brain areas were assayed for levels of dopamine (DA), 3,4-dihydroxyphenylacetic acid (DOPAC), homovanillic acid (HVA), 5-HT, and 5-hydroxyindoleacetic acid (5-HIAA) by using high-performance liquid chromatography (HPLC) with electrochemical detection as previously described in Matsuda, T., Seong, Y. H., Aono, H., Kanda, T., Baba, A., Saito, K., Tobe, A. & Iwata, H. (1989) Eur. J. Pharmacol. 170, 75-82.

(2) Results

(2-1) Characterization of PACAP−/− Mice

The mouse PACAP gene (Yamamoto, K., Hashimoto, H., Hagihara, N., Nishino, A., Fujita, T., Matsuda, T. & Baba, A. (1998) Gene 211, 63-69) was disrupted in embryonic stem (ES) cells by homologous recombination through inactivation of part of exon 5, from which the mature PACAP (PACAP38 and PACAP27) protein is expressed (FIG. 1A). Four positive ES clones were obtained and two of them were selected and used to generate mutant mice, whose genotypes were confirmed by Southern hybridization (FIG. 1B). In situ hybridization analysis with a probe corresponding to the disrupted region indicated that the expression of the mature peptide coding sequence was greatly reduced in heterozygous (PACAP+/−) mice compared with wild-type mice and disappeared in PACAP−/− mice (FIG. 1C). The complete deletion of this sequence was also confirmed by RT-PCR (FIG. 1D). In addition, expression of the upstream coding sequence was reduced (FIG. 1D) in a gene dose-dependent manner (FIG. 1C). The absence of PACAP38 expression in the brain (midbrain and diencephalon) of PACAP−/− mice was demonstrated by radioimmunoassay; furthermore, PACAP38 expression was 2S greatly reduced in PACAP+/−mice compared with wild-type mice (FIG. 1E).

Mendelian segregation of pup genotypes from heterozygous breeding (n=117) was observed at birth, with a genotype distribution of 23.9%, 49.6%, and 26.5% for PACAP−/−, PACAP+/−, and wild-type mice, respectively. However, PACAP−/− pups had a high mortality rate, and the genotype distribution upon weaning (n=531) was 15.6%, 54.8%, and 29.6% for PACAP−/−, PACAP+/−, and wild-type mice, respectively, suggesting a significant loss of PACAP−/− pups (p <0.001, %2 analysis). Homozygous mating of PACAP-deficient mice resulted in fewer successful pregnancies, yielding few pups (data not shown). PACAP−/− female mice could nurse their newborn pups, although PACAP−/− pups had a high mortality rate, irrespective of their parent genotypes.

Hyperactivity in a Novel Environment

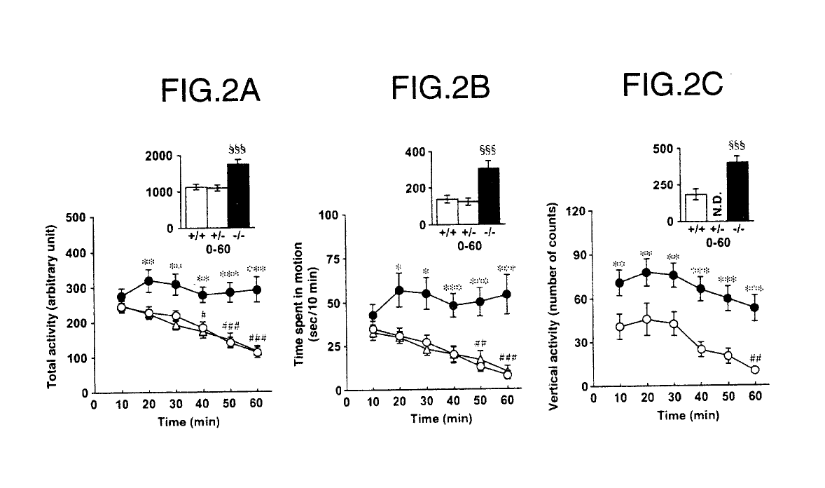

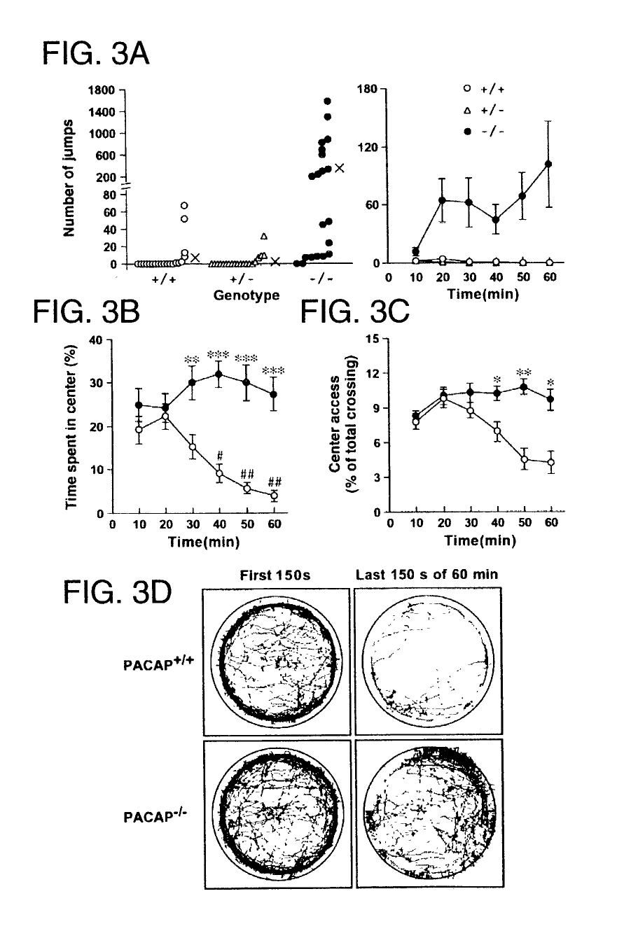

The present inventors analyzed the behavior of PACAP−/−mice in an open field to assess their locomotor activity in novel environments. As shown in FIGS. 2A and B, PACAP−/− mice exhibited higher levels of locomotor activity (total activity), spent more time in motion than wild-type mice, and showed minimal habituation to the novel environment for 60 min, at which time wild-type mice were habituated significantly. Tracks of locomotor patterns during the first and last 150 s clearly show that, although the initial levels of locomotion were similar for both genotypes, PACAP−/− mice were more active than wild-type mice at the end of the session (FIG. 3D). Analysis of the mean speed of motion revealed hyperkinetic movement of PACAP−/− mice (PACAP+/+, 20.0±0.59 cm/s; PACAP−/−, 23.7±0.66 cm/s, p<0.001, Mann-Whitney test). Furthermore, PACAP−/− mice spent less time engaged in licking/grooming behaviors (PACAP+/+, 19.7±2.5%; PACAP−/−, 10.0±1.9%, p<0.001, Mann-Whitney test). There was no difference between PACAP+/−mice and wild-type mice in behavioral measures in the open field (FIGS. 2A, B). Vertical activity, including rearing and jumping (see below), was much higher in PACAP−/− mice than in wild-type mice, indicating increased exploratory activity (FIG. 2C). PACAP−/− mice which have ˜97% of ICR genetic background similarly showed higher levels of locomotor activity than wild-type littermate control mice (p<0.01) or wild-type inbred ICR mice (p<0.001) in the open field (total activity for 90 mm in arbitrary unit, n=10-11: PACAP−/−, 1896±84; PACAP+/+, 1435±101; inbred ICR, 1322±63).

Interestingly, PACAP−/− mice elicited explosive jumping behavior in the open field arena, which was only rarely observed in wild-type or PACAP+/− mice (FIGS. 3A, D). In almost all of PACAP−/− mice, the frequency of jumping escalated over time, with a maximum of more than 1500 times during a 60-min observation period. PACAP−/− mice with ICR background again exhibited explosive jumping behavior in the open field more frequently than wild-type littermate controls (p<0.001) or wild-type inbred ICR mice (p<0.01) (number of jumps for 90 min, n=10-11: PACAP−/−, 225±49; PACAP+/+, 21±8; inbred ICR, 30±18).

(2-2) Increased Exploration and Reduced Anxiety

Increased exploratory-related and reduced anxiety-related behavior in PACAP−/− mice was demonstrated by zone monitoring in the open field, on the elevated plus maze, and by the emergence and novel object tests. In the open field, PACAP−/− mice entered the center region more often and spent more time in the center than wild-type mice (FIGS. 3B-D). When tested on an elevated plus maze, PACAP−/− mice showed a tendency to enter the open arms of the maze more often than wild-type controls. They also spent much more time on the open arms than wild-type controls (Table 1). The emergence test is a free exploration paradigm designed to reduce anxiety in mice confronted with a novel open field environment with no possibility of escape by providing a safe enclosure in order to assess approach or exploratory behavior in rodents (Dulawa, S. C., Grandy, D. K., Low, M. J., Paulus, M. P. & Geyer, M. A. (1999) J. Neurosci. 19, 9550-9556). PACAP−/− mice exhibited shortened latencies for emergence and decreased total time spent in the cylinder, and demonstrated increased exploratory behavior (assessed by frequency of rearing to the wall of the cylinder) compared with wild-type mice (Table 1). The novel object test is another free exploration paradigm that provides animals the opportunity to explore a novel object (Dulawa, S. C., Grandy, D. K., Low, M. J., Paulus, M. P. & Geyer, M. A. (1999) J. Neurosci. 19, 9550-9556). Upon introduction of the novel object (a white cup), PACAP−/− mice increased the frequency of center access and time spent in the center more than wild-type controls (Table 1). Thus, PACAP−/− mice exhibited elevated behavioral responses to novelty with increased exploration and less anxiety compared to wild-type controls.

(2-3) Effetcs of Haloperidol on Hyperactive Behavior and Catalepsy

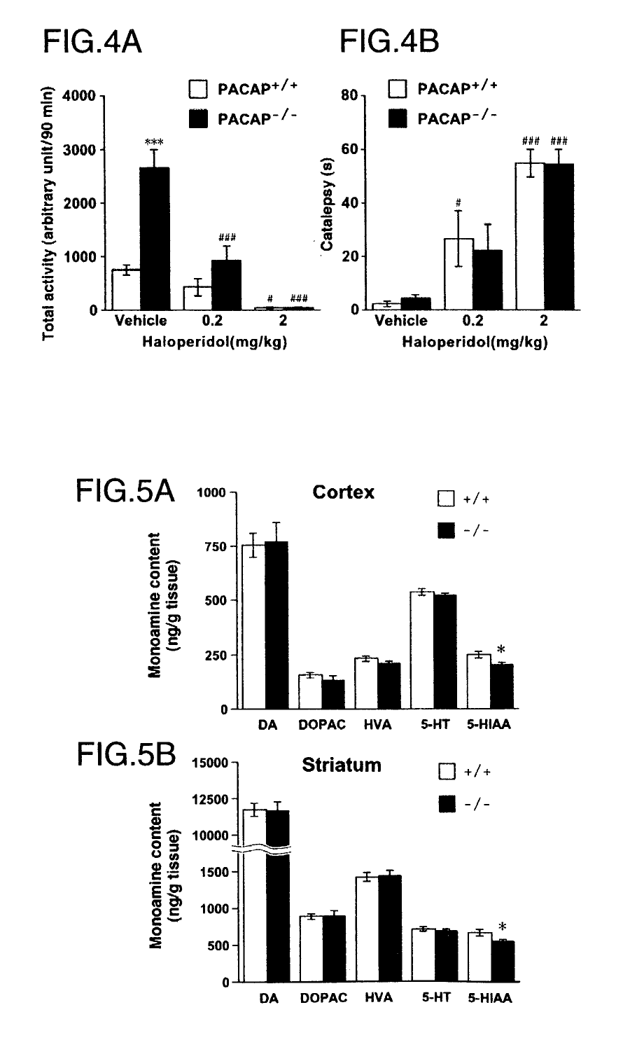

The antipsychotic drug haloperidol was tested for its ability to attenuate the hyperactive behavior of PACAP−/− mice (FIG. 4). Haloperidol (0.2 mg/kg) effectively reduced the hyperactivity in PACAP−/− mice (65±10% reduction) to the control level in wild-type mice, although the same dose of haloperidol resulted in moderate impairment of locomotor activity in wild-type mice (43±21% reduction) (FIG. 4A). PACAP−/− mice did not show spontaneous catalepsy, and the cataleptogenic effect of haloperidol (0.2 and 2 mg/kg) in PACAP−/− mice was quite similar to that in wild-type mice, suggesting that the extrapyramidal system (postsynaptic striatal DA receptors) of PACAP−/− mice is functionally normal (FIG. 4B).

(2-4) Analyses of Monoamine Turnover in PACAP−/− Mice

The effect of PACAP deficiency on monoamine turnover was determined by HPLC (FIG. 5). In the cortex and striatum, the 5-HIAA levels were slightly but statistically lower in PACAP−/− mice compared with wild-type mice (81% and 82%, respectively). The cortical and striatal 5-HIAA/5-HT ratios were reduced by 15% in PACAP−/− mice. No significant differences in levels of monoamines and their metabolites have, so far, been observed in any other brain regions between wild-type and PACAP−/− mice (data not shown).

(3) Discussion

The results of behavioral experiments with PACAP−/− mice demonstrate that disruption of the PACAP gene in mice lead to perturbations in psychomotor behaviors, especially the exploratory component of locomotor behavior, implicating PACAP in psychotic brain functions. Furthermore, the 5-HIAA level was decreased slightly in the cortex and striatum of the PACAP−/− mouse brain.

It is commonly believed that locomotor hyperactivity is associated with increased DA tone (Gainetdinov, R. R., Wetsel, W. C., Jones, S. R., Levin, E. D., Jaber, M. & Caron, M. G. (1999) Science 283, 397-401). In PACAP−/− mice, DA turnover was unchanged and the incidence of haloperidol-induced catalepsy was quite similar to that in wild-type mice. These findings suggest that the locomotor hyperactivity in PACAP−/− mice may probably not a result of increased nigrostriatal dopaminergic activity. Alternatively, the importance of 5-HT in controlling the locomotor activity has been demonstrated in a study using 5-HT1B receptor knockout mice (Saudou, F., Amara, D. A., Dierich, A., LeMeur, M., Ramboz, S., Segu, L., Buhot, M. C. & Hen, R. (1994) Science 265, 1875-1878). Moreover, a relative balance of the DA and 5-HT systems seems to be important for normal motor activity, and alterations in any of the parameters that control this delicate homeostatic situation might underlie hyperactive states (Gainetdinov, R. R., Wetsel, W. C., Jones, S. R., Levin, E. D., Jaber, M. & Caron, M. G. (1999) Science 283, 397-401). This may explain, at least in part, the locomotor hyperactivity of PACAP−/− mice.

Several lines of evidence suggest that dysfunction of serotonergic pathways, especially those mediated by the 5-HT1A receptor, is associated with anxiety-related traits (Hoyer, D., Clarke, D. E., Fozard, J. R., Hartig, P. R., Martin, G. R., Mylecharane, E. J., Saxena, P. R. & Humphrey, P. P. (1994) Pharmacol. Rev. 46, 157-203). Although it is suggested that PACAP−/− mice are less anxious, reduced 5-HT turnover (5-HIAA/5-HT ratio) cannot explain it. On the other hand, DA functions, particularly those mediated by the D4 receptor, are involved in novelty-related exploratory behavior, as reported in D4 receptor knockout mice (Dulawa, S. C., Grandy, D. K., Low, M. J., Paulus, M. P. & Geyer, M. A. (1999) J. Neurosci. 19, 9550-9556; Rubinstein, M., Phillips, T. J., Bunzow, J. R., Falzone, T. L., Dziewczapolski, G., Zhang, G., Fang, Y., Larson, J. L., McDougall, J. A., Chester, J. A., Saez, C., Pugsley, T. A., Gershanik, O., Low, M. J. & Grandy, D. K. (1997) Cell 90, 991-1001). This mutant mice are less active in open field tests and exhibit reduced exploration of novel stimuli in contrast to the phenotypes observed in PACAP−/− mice.

One of the striking findings of the present study was that PACAP−/− mice showed abnormal jumping behavior in the open field arena. In NIH Swiss mice, an NMDA receptor antagonist MK-801 is known to precipitate explosive episodic jumping behavior which can be attenuated by haloperidol (Rosse, R. B., Mastropaolo, J., Sussman, D. M., Koetzner, L., Morn, C. B. & Deutsch, S. I. (1995) Clin. Neuropharmacol. 18, 448-457). In addition, dysfunction of the NMDA receptor by MK-801 or the targeted disruption of its gene produces psychotic symptoms which closely resemble the positive and negative symptoms of schizophrenia (Mohn, A. R., Gainetdinov, R. R., Caron, M. G. & Koller, B. H. (1999) Cell 98, 427-436; Svensson, T. H. (2000) Brain Res. Brain Res. Rev. 31, 320-329). In view of the effects of MK-801, investigation of possible common mechanisms involved in the MK-801-induced abnormal behaviors and those of PACAP−/− mice would be warranted.

It is currently accepted that multiple genes of small effect, rather than a single causative gene, act in concert with non-genetic factors to increase the risk of mental disorder (Hyman, S. E. (2000) Bull. World Health Organ. 78, 455-463). The present study shows that PACAP−/− mice display marked behavioral abnormalities without having marked changes in specific gene expression in their brains. To date, The present inventors have found no change at the level of gene expression of PACAP receptor subtypes, tyrosine hydroxylase, and DA D2 receptor in the various brain regions of PACAP−/− mice (data not shown). Of course, there are many other functional molecules for which expression levels should be determined. It is possible that PACAP−/− mice have several small but significant changes in the activity of neuronal networks and that they collectively cause behavioral abnormalities.

Because specific low-molecular weight antagonists and agonists to the different PACAP receptor subtypes are not available, the physiological role of PACAP as well as of each receptor subtype in brain function have not been fully addressed. PAC1 receptor-deficient mice (Hashimoto, H., Shintani, N., Nishino, A., Okabe, M., Ikawa, M., Matsuyama, S., Itoh, K., Yamamoto, K., Tomimoto, S., Fujita, T., Hagihara, N., Mori, W., Koyama, Y., Matsuda, T., Nagata, S. & Baba, A. (2000) J. Neurochem. 75, 1810-1817; Jamen, F., Persson, K., Bertrand, G., Rodriguez Henche, N., Puech, R., Bockaert, J., Ahren, B. & Brabet, P. (2000) J. Clin. Invest. 105, 1307-1315; Sauvage, M., Brabet, P., Holsboer, F., Bockaert, J. & Steckler, T. (2000) Brain Res. Mol. Brain Res. 84, 79-89) did not show any apparent behavioral changes like those observed in PACAP−/− mice. PACAP interacts with three receptors-PACA -preferring PAC1 and VIP-shared VPAC1 and VPAC2 receptors. Lack of signal transmission of PACAP through the VPAC receptors may explain the behavioral changes in PACAP−/− mice.

Several lines of evidence suggest that PACAP acts as a neurotrophic factor (Takei, N., Skoglosa, Y. & Lindholm, D. (1998) J. Neurosci. Res. 54, 698-706; Morio, H., Tatsuno, I., Hirai, A., Tamura, Y. & Saito, Y. (1996) Brain Res. 741, 82-88; Villalba, M., Bockaert, J. & Journot, L. (1997) J. Neurosci. 17, 83-90) and plays a role in mammalian neurogenesis (Jaworski, D. M. & Proctor, M. D. (2000) Brain Res. Dev. Brain Res. 120, 27-39). For instance, the PACI receptor is expressed at very high levels in ventricular zones throughout the embryonic neuraxis, and PACAP likely regulates the development of the general features of the neuronal phenotype (Jaworski, D. M. & Proctor, M. D. (2000) Brain Res. Dev. Brain Res. 120, 27-39). Therefore, it is also possible that the lack of PACAP affects developmental processes, resulting in the observed behavioral abnormalities and in a high early mortality rate in PACAP−/− mice.

Since it was demonstrated that the Drosophila mutant amnesiac, which displays behavioral defects, has a mutation in a neuropeptide gene, the in vivo role of the mammalian homolog PACAP has remained an open question (Feany, M. B. & Quinn, W. G. (1995) Science 268, 869-873). In this study, The present inventors have shown that disruption of the PACAP gene in mice leads to major alterations in psychomotor activity. Recent genetic linkage studies have suggested that a locus for schizophrenia as well as bipolar affective disorder is located on chromosome 18p11 (Rojas, K., Liang, L., Johnson, E. I., Berrettini, W. H. & Overhauser, J. (2000) Mol. Psychiatry 5, 389-395), where the human PACAP gene resides (Hosoya, M., Kimura, C., Ogi, K., Ohkubo, S., Miyamoto, Y., Kugoh, H., Shimizu, M., Onda, H., Oshimura, M., Arimura, A. & Fujino, M. (1992) Biochim. Biophys. Acta 1129, 199-206). It is now important to determine whether the mutation in the PACAP locus is implicated in disease in these select families.

In conclusion, the present study proposes a role of PACAP-ergic neurons in regulating psychomotor behaviors acutely or developmentally. The PACAP−/− mouse should be a valuable tool to investigate both normal and pathological processes in which PACAP has been proposed to play a role.

Figure Ledends

FIGS. 1A-1E

Targeted disruption of the PACAP gene and characterization of PACAP−/−, PACAP+/−, and wild-type mice. (A) Alignment of the PACAP locus with the targeting vector and the mutant locus. Closed boxes 1A through 5: exons 1A through 5; DT: MC1 promoter with diphtheria toxin A-fragment gene; neo: phosphoglycerate kinase promoter with neomycin resistance gene. (B) Southern blot analysis of tail DNA digested either with XhoI and SphI and hybridized with a 5′ probe (top) or with HindIII and hybridized with a 3′ probe (bottom). (C) In situ hybridization analysis of parasagittal brain sections with two 35S-cRNA probes specific for exons 2-4 and exon 5. Scale bar, 2.5 mm. (D) RT-PCR analysis of RNA from midbrain and diencephalon. PCR was performed with the indicated number of cycles using a sense primer derived from PACAP exon 3 and antisense primers derived from PACAP exon 4 (left) and exon 5 (center). The β-actin housekeeping gene was simultaneously amplified as an internal standard (right). (E) Analysis of PACAP38 levels in the midbrain and diencephalon from PACAP+/+ (n=7), PACAP+/− (n=6) and PACAP−/− (n=5) mice by radioimmunoassay. ***p<0.001 versus PACAP+/+mice; ###p<0.001 versus PACAP+/−mice, Student's t test.

FIGS. 2A-2C

Open field measures. (A and B) Total activity (A) and time spent in motion (B) are shown for PACAP+/+ (open circles), PACAP+/−(open triangles), and PACAP−/− (closed circles) mice (n=20 for all groups). (C) Vertical activity is shown for PACAP+/+and PACAP−/− mice (n=14 for both groups). *p<0.05, **p<0.01 and ***p<0.001 versus PACAP+/+mice, #p<0.05, ##p<0.01 and ###p<0.001 versus first 10 min, ANOVA, followed by post hoc Fisher PLSD test; §§§p<0.001 versus PACAP+/+mice, Kruskal-Wallis ANOVA, followed by Mann-Whitney test. N.D., not determined.

FIGS. 3A-3D

Jumping behavior, increased central entry, and locomotor pattern of PACAP−/− mice in the open field. (A) Number of jumps in the open field. The actual number of jumps observed in individual mice and mean values (crosses) for 60 min (left), and means ±s.e. at 10-min intervals (right) are indicated. n=20 for all groups. (B and C) Time spent in the center (B) and center access (percentage of crossings into the center zone out of the total number of crossings) (C) are shown for PACAP+/+ (open circles) and PACAP−/− (closed circles) mice (n=20 for all groups). *p<0.05, **p<0.01 and ***p<0.001 versus PACAP+/+ mice, #p<0.05 and ##p<0.01 versus first 10 min, ANOVA, followed by post hoc Fisher PLSD test. (D) Examples of locomotor patterns of PACAP+/+ (top) and PACAP−/− (bottom) mice during the first 150 sec (left) and last 150 sec (right) of a 60-min recording. Tracks of representative 10 mice in each group are shown. The tracks outward across the circular boundary represent the jumping behavior.

FIGS. 4A-4B

Effects of haloperidol on locomotor activity. (A) PACAP+/+and PACAP−/− mice (n =6-7) were injected intraperitoneally with 0.2 and 2 mg/kg haloperidol or vehicle (0.5% carboxymethyl cellulose sodium) and 20 min later placed in the open field for automated measurement of locomotor parameters over a 90 min test period. (B) Subsequently, each mouse was tested for catalepsy in the bar test. Values are means±s.e. ***p<0.001 versus PACAP+/+ mice, #p<0.05 and ###p<0.001 versus vehicle, ANOVA, followed by post hoc Fisher PLSD test.

FIGS. 5A-5B

Tissue content of monoamines and their metabolites in cortex and striatum.

Levels of the monoamine neurotransmitters and the major metabolites were assayed in cortex (A) and striatum (B) of PACAP+/+ (n=14) and PACAP−/− (n=13) mice. Data are expressed as means±s.e. *p<0.05 versus PACAP+/+ mice, Student's t test.

TABLE 1
Increased exploratory activity and reduced
anxiety-related behavior of PACAP⁻/⁻mice as
revealed by the elevated plus-maze, emergence,
and novel object tests

| | | | |
|---|---|---|---|
| Test | PACAP⁺/⁺ | PACAP⁺/⁻ | PACAP⁻/⁻ |

Elevated plus-maze (n = 7-8 mice per genotype)

| | | | |
|---|---|---|---|
| Total path length (cm/5 min) | 747 ± 109 | 902 ± 113 | 1158 ± 83* |
| Open arm entries (%) | 24.3 ± 7.1 | 18.8 ± 6.5 | 37.0 ± 4.3# |
| Time spent in open arms (%) | 3.2 ± 1.4 | 9.6 ± 5.7 | 20.1 ± 2.0**,# |

Emergence test (n = 16 mice per genotype)

| | | | |
|---|---|---|---|
| Latency of emergence (s) | 888 ± 12 | — | 587 ± 71*** |
| Time spent in the cylinder (s) | 888 ± 12 | — | 600 ± 69** |
| Exploratory behavior | 16.0 ± 3.5 | — | 30.8 ± 2.7** |

Novel object test (n = 8 mice per genotype)
Number of center access

| | | | |
|---|---|---|---|
| No cup | 13.6 ± 4.6 | — | 13.5 ± 4.3 |
| cup | 6.5 ± 3.4 | — | 30.3 ± 5.2**, § |

Time spent in the center region (s)

| | | | |
|---|---|---|---|
| No cup | 11.7 ± 4.6 | — | 18.2 ± 8.2 |
| cup | 13.2 ± 7.1 | — | 79.9 ± 24.6**, § |

Values are means ± s.e. *p < 0.05, **p < 0.01, and ***p < 0.001 versus PACAP⁺/⁺group; #p < 0.05 versus PACAP⁺/⁻group; §p < 0.05 versus no cup, Kruskal-Wallis ANOVA, followed by Mann-Whitney test (elevated plus-maze and novel object tests) and Mann-Whitney test (emergence test).

What is claimed is:

1. A mammalian model animal for psychiatric disorders having a chromosome of a somatic cell and a germ cell with deficiency of function of pituitary adenylate cyclase-activating polypeptide gene.

2. The mammalian model animal according to claim 1, wherein said function is defective due to deficiency of a part or whole of exon 5 in said pituitary adenylate cyclase-activating polypeptide gene.

3. The mammalian model animal according to claim 1, wherein said function is defective due to introducing a point mutation or inserting another gene in exon 5.

4. The mammalian model animal according to claim 2, wherein a part or whole of exon 5 is deleted by substituting the part or whole of the exon 5 by another gene.

5. The mammalian model animal according to claim 4, wherein said another gene is a marker gene.

6. The mammalian model animal according to claim 5, wherein said marker gene is a neomycin resistance gene.

7. The mammalian model animal according to claim 1, wherein the mammalian animal is a rodent.

8. The mammalian model animal according to claim 1, wherein the mammalian animal is a mouse.
